(19) United States
(12) Patent Application Publication
(10) Pub. No.: US 2014/0234913 A1
(43) Pub. Date: Aug. 21, 2014

(54) PROCESS FOR TREATING A SUBSTRATE WITH AN ENZYME

(71) Applicant: Novozymes A/S, Bagsvaerd (DK)

(72) Inventor: Ole Simonsen, Soeborg (DK)

(73) Assignee: Novozymes A/S, Bagsvaerd (DK)

(21) Appl. No.: 14/168,507

(22) Filed: Jan. 30, 2014

Related U.S. Application Data
(63) Continuation of application No. 13/377,844, filed on Dec. 13, 2011, now abandoned.

(30) Foreign Application Priority Data
Jul. 7, 2009 (EP) 09164787.5

(51) Int. Cl. C12P 7/06; C12P 19/14

(52) U.S. Cl. CPC C12P 7/06; C12P 19/14; C12P 2203/00
USPC 435/99; 435/161

(57) ABSTRACT

In a process for hydrolyzing plant material in aqueous solution or suspension with an enzyme, the enzyme is delivered in solid form (e.g., as a spray-dried powder) in closed containers (such as paper bags or cardboard boxes), which are added directly in the process (i.e., addition of whole boxes/bags). The invention is particularly amenable to the production of first or second-generation bioethanol.

### CROSS-REFERENCE TO RELATED APPLICATIONS

This application is a continuation of U.S. application Ser. No. 13/377,844 filed on Dec. 13, 2011, now pending, which is a 35 U.S.C. 371 national application of PCT/EP2010/059731 filed on Jul. 7, 2010, which claims priority or the benefit under 35 U.S.C. 119 of European application No. 09164787.5 filed on Jul. 7, 2009, the contents of which are fully incorporated herein by reference.

### FIELD OF THE INVENTION

The present invention relates to a process for hydrolyzing plant material in aqueous solution or suspension with an enzyme.

### BACKGROUND OF THE INVENTION

Enzymatic hydrolysis of plant material in aqueous solution or suspension is widely used, and it may involve the addition of a single enzyme or the simultaneous addition of two or more enzymes. One example is the treatment of starch-containing raw materials with starch-hydrolyzing enzymes such as alpha-amylase and glucoamylase for the production of first-generation bioethanol. Another example is the treatment of lignocellulosic biomass with enzymes such as cellulases and hemicellulases for the production of second-generation bioethanol.

Typically, the enzyme is produced in one location, and the treatment of plant material takes place in a different location, so the enzyme needs to be transported and to be held for some time. If the enzyme is in liquid form, it is usually necessary to add a stabilizer such as a polyol, thus increasing the production cost, and the weight of water and stabilizer increase the transportation and handling costs. It is attractive to use solid products as they offer better stability, and contain less or no water and hence have reduced transportation costs, and enzymes in solid form may be produced at moderate cost, e.g., by spray drying and/or fluid bed drying. However, spray-dried powders have some serious draw-backs with regard to safety in handling and flowability of the cohesive powders. Further it is difficult and expensive to make homogenous blends of cohesive powders. Alternatively, the enzymes may be provided as low-dusting granulates, but granulation adds to the cost.

### SUMMARY OF THE INVENTION

The above-mentioned draw-backs can be overcome by delivering the enzyme in solid form (e.g., as a spray-dried powder) in closed containers (such as paper bags or cardboard boxes), which are added directly in the process (i.e., addition of whole boxes/bags) to the solution or suspension of the plant material. The enzyme dissolves upon contact with the aqueous solution or suspension, and the container may become permeable in wet form, or it may dissolve or disintegrate due to wetting (e.g., PVA or paper bags or cardboard boxes) or to mechanical action of an agitator. Also, the material of the container (e.g., cellulose in paper cardboard) may be broken down by the action of the enzyme (e.g., cellulase).

Addition of closed containers will minimize the dust formation during handling, and it is possible to fill the containers with different enzyme powder, without having to homogenize the powders.

Accordingly, the invention provides a process for hydrolyzing plant material, comprising:

a) preparing an aqueous solution or suspension of the plant material,

b) adding one or more container(s) which enclose(s) a multitude of particles comprising one or more enzyme(s) having hydrolytic activity towards the plant material so that the enzyme(s) is/are released to the solution or suspension, and

c) incubating the solution or suspension so as to hydrolyze the plant material.

Furthermore, the invention provides a container for use in the process which comprises a multitude of particles having glucoamylase activity.

### DETAILED DESCRIPTION OF THE INVENTION

### Hydrolysis of Plant Material

The invention is particularly amenable to the production of first or second-generation bioethanol. Thus, the plant material may particularly comprise lignocellulosic biomass, or it may comprise starch-containing material.

Optionally, the hydrolyzed plant material may be fermented by adding a fermenting organism (such as yeast) and incubating so as to form a fermentation product during or after the hydrolysis step. The fermentation product may particularly be biofuels products such as ethanol and butanol. The fermentation may be carried out at conventionally used conditions. Preferred fermentation processes are anaerobic processes.

For ethanol production the fermentation may in one embodiment go on for 6 to 120 hours, in particular 24 to 96 hours. In an embodiment the fermentation is carried out at a temperature between 25 to 40° C., preferably 28 to 35° C., such as 30 to 34° C., and in particular around 32° C. In an embodiment the pH when initiating fermentation is in the range from pH 3 to 6, preferably around pH 4 to 5.

After fermentation the fermenting organism may be separated from the fermented slurry and recycled to the fermentation medium.

Subsequent to fermentation the fermentation product may be separated from the fermentation medium. The fermented solution or slurry may be distilled to extract the desired fermentation product, or the desired fermentation product may be extracted from the fermentation medium by micro or membrane filtration techniques. Alternatively the fermentation product may be recovered by stripping. Methods for recovery are well known in the art.

The substrate solution or suspension may have a volume of at least 1 m³, particularly at least 5 m³, at least 10 m³ or at least 25 m³. Thus. the substrate solution or suspension may be contained in a tank or vessel having said volume.

### Enzyme Particles

The enzyme particles typically include enzymes made by fermenting a microorganism, and they may be produced by spray drying and/or fluid bed drying. Before drying, the fermentation broth may be sterilized to kill living microbial cells and/or purified to remove biomass, e.g., by filtration, centrifugation, and/or flocculation. For cost saving reasons the broth including microbial cells and/or cell debris may be dried directly, e.g., as described in WO 01/25411.

The enzyme particles (e.g., spray-dried powder) typically have an average particle size (weight average) below 2 mm, e.g., in the range 5-200 μm. The enzyme particles typically have an average mass below 1 g, particularly below 100 mg, below 10 mg or below 1 mg.

The enzyme with hydrolytic activity towards the plant material is a hydrolase in class EC 3.-.-.-). EC numbers are defined in the handbook Enzyme Nomenclature from NC-IUBMB, 1992), or on the ENZYME site at the internet: www.expasy.ch/enzyme.

### Container

The containers for the enzyme particles may be bags or boxes made of water soluble or dispersible packaging material, e.g., paper, cardboard, polyvinyl alcohol, or water soluble cellulose or starch derivatives. Double containers may be used, e.g., paper bags in cardboard boxes. The containers are added to the substrate solution or suspension in closed form, to avoid dust formation.

Each container filled with enzyme particles will typically have a mass of at least 1 kg, e.g., at least 5 kg, at least 10 kg, at least 15 kg, at least 20 kg, at least 25 kg, at least 100 kg or at least 500 kg.

The container contains enzyme-containing particles, and optionally it may also contain enzymatically inert particles, e.g., other components useful for the process, e.g., enzyme cofactors as calcium or other divalent cations, e.g., in an amount below 75% by weight, particularly below 50% or below 25%. The enzyme particles may comprise at least 1% w/w of enzyme protein, particularly at least 5%, at least 10%, at least 20%, at least 30%, at least 40%, at least 50%, at least 60%, at least 70%, at least 75%, at least 80% or at least 90%.

### Starch Hydrolysis

In the case of plant material comprising starch, the hydrolase may be selected among carbohydrases, e.g., glycosidases (EC 3.2), such as alpha-amylases (EC 3.2.1.1) and glucan 1,4-alpha-glucosidases (glucoamylase; amyloglucosidase, EC 3.2.1.3). In particular, the enzyme particles may comprise glucoamylase at an activity of at least 0.1 AGU/g and/or alpha-amylase at an activity of at least 0.02 FAU-F/g. The AGU unit is defined in WO 04/080923 and the FAU-F unit in WO 2009/094614. The alpha-amylase may be bacterial or fungal.

### Liquefaction and Saccharification of Starch-Containing Material

Starch-containing plant material may be treated by liquefaction with an alpha-amylase, followed by saccharification with a glucoamylase and optionally fermentation with a fermenting organism (such as yeast), e.g., as described in WO 96/28567. The saccharification and the fermentation may be performed sequentially with a separate holding stage for the saccharification, or they may be simultaneous, meaning that the saccharifying enzyme(s) and the fermenting organism may be added together. When fermentation is performed simultaneous with hydrolysis/saccharification the temperature is preferably between 25 to 40° C., preferably 28 to 35° C., such as 30 to 34° C., in particular around 32° C., when the fermentation organism is a strain of Saccharomyces cerevisiae and the desired fermentation product is ethanol.

Other fermentation products may be fermented at temperatures known to the skilled person in the art to be suitable for the fermenting organism in question.

The fermentation product, such as especially ethanol, may optionally be recovered after fermentation, e.g., by distillation. The liquefaction is preferably carried out in the presence of an alpha-amylase, preferably a bacterial alpha-amylase or acid fungal alpha-amylase. The fermenting organism is preferably yeast, preferably a strain of Saccharomyces.

In a particular embodiment, the process further comprises, prior to the step (i), the steps of:

x) reducing the particle size of the starch-containing material, preferably by milling;

y) forming a slurry comprising the starch-containing material and water.

The aqueous slurry may contain from 10-55 wt. % dry solids (DS), preferably 25-45 wt. % dry solids, more preferably 30-40 wt. % dry solids of starch-containing material. The slurry is heated to above the gelatinization temperature and alpha-amylase, preferably bacterial and/or acid fungal alpha-amylase may be added to initiate liquefaction (thinning). The slurry may in an embodiment be jet-cooked to further gelatinize the slurry before being subjected to an alpha-amylase in step (i).

### Production of Fermentation Products From Un-Gelatinized Starch-Containing Material

A fermentation product may be produced from starch-containing material without gelatinization (often referred to as “cooking”) of the starch-containing material, e.g., as described in U.S. Pat. No. 4,316,956. The desired fermentation product, such as ethanol, can be produced without liquefying the aqueous slurry containing the starch-containing material. In one embodiment a process includes saccharifying (e.g., milled) starch-containing material, e.g., granular starch, below the initial gelatinization temperature, preferably in the presence of an alpha-amylase and/or an carbohydrate-source generating enzyme to produce sugars that can be fermented into the desired fermentation product by a suitable fermenting organism.

In this embodiment the desired fermentation product, preferably ethanol, is produced from ungelatinized (i.e., uncooked), preferably milled corn. Accordingly, this aspect relates to a process of producing a fermentation product from starch-containing material, comprising the steps of:

(a) saccharifying starch-containing material at a temperature below the initial gelatinization temperature of said starch-containing material,

(b) fermenting using a fermenting organism.

Steps (a) and (b) may be carried out simultaneously (i.e., one step fermentation) or sequentially. The process may be performed as a batch or as a continuous process. The fermentation process may be conducted in an ultrafiltration system where the retentate is held under recirculation in the presence of solids, water, and the fermenting organism, and where the permeate is the desired fermentation product containing liquid. Equally contemplated if the process is conducted in a continuous membrane reactor with ultrafiltration membranes and where the retentate is held under recirculation in presence of solids, water, the fermenting organism and where the permeate is the fermentation product containing liquid.

### Bacterial Alpha-Amylases

The bacterial alpha-amylase may be derived from the genus Bacillus, e.g., from a strain of Bacillus licheniformis, Bacillus amyloliquefaciens, Bacillus subtilis or Bacillus stearothermophilus. Specific examples include the Bacillus licheniformis alpha-amylase shown in SEQ ID NO: 4 in WO 99/19467, the Bacillus amyloliquefaciens alpha-amylase shown in SEQ ID NO: 5 in WO 99/19467 and the Bacillus stearothermophilus alpha-amylase shown in SEQ ID NO: 3 in WO 99/19467 (all sequences hereby incorporated by reference).

### Fungal Alpha-Amylases

Fungal alpha-amylases include alpha-amylases derived from a strain of the genus Aspergillus, such as, Aspergillus oryzae, Aspergillus niger and Aspergillis kawachii alpha-amylases.

A preferred acidic fungal alpha-amylase is a Fungamyl-like alpha-amylase which is derived from a strain of Aspergillus oryzae which exhibits a high identity, i.e., at least 70%, at least 75%, at least 80%, at least 85%, at least 90%, at least 95%, at least 96%, at least 97%, at least 98%, at least 99% or even 100% identity to the mature part of the amino acid sequence shown in SEQ ID NO: 10 in WO 96/23874.

Another preferred acid alpha-amylase is derived from a strain Aspergillus niger. In a preferred embodiment the acid fungal alpha-amylase is the one from Aspergillus niger disclosed as “AMYA_ASPNG” in the Swiss-prot/TeEMBL database under the primary accession no. P56271 and described in WO 89/01969 (Example 3—incorporated by reference). A commercially available acid fungal alpha-amylase derived from Aspergillus niger is SP288 (available from Novozymes NS, Denmark).

Other contemplated wild-type alpha-amylases include those derived from a strain of the genera Rhizomucor and Meripilus, preferably a strain of Rhizomucor pusillus (WO 2004/055178 incorporated by reference) or Meripilus giganteus.

In a preferred embodiment the alpha-amylase is derived from Aspergillus kawachii and disclosed by Kaneko et al., 1996, “Molecular-cloning and determination of the nucleotide-sequence of a gene encoding an acid-stable alpha-amylase from Aspergillus kawachii”, J. Ferment. Bioeng. 81: 292-298 and further as EMBL:#AB008370.

The fungal alpha-amylase may also be a wild-type enzyme comprising a starch-binding domain (SBD) and an alpha-amylase catalytic domain (i.e., non-hybrid), or a variant thereof. In an embodiment the wild-type alpha-amylase is derived from a strain of Aspergillus kawachii.

### Glucoamylases

A glucoamylase may be derived from any suitable source, e.g., derived from a microorganism or a plant. Preferred glucoamylases are of fungal or bacterial origin, e.g., selected from the group consisting of Aspergillus glucoamylases, in particular Aspergillus niger G1 or G2 glucoamylase (Boel et al., 1984, EMBO J. 3(5): 1097-1102), or variants thereof, such as those disclosed in WO 92/00381, WO 00/04136 and WO 01/04273 (from Novozymes, Denmark); the A. awamori glucoamylase disclosed in WO 84/02921, Aspergillus oryzae glucoamylase (Agric. Biol. Chem. 55(4): 941-949 (,1991)), or variants or fragments thereof. Other Aspergillus glucoamylase variants include variants with enhanced thermal stability: G137A and G139A (Chen et al., 1996, Prot. Eng. 9: 499-505); D257E and D293E/Q (Chen et al., 1995, Prot. Eng. 8: 575-582); N182 (Chen et al., 1994, Biochem. J. 301: 275-281); disulphide bonds, A246C (Fierobe et al., 1996, Biochemistry 35: 8698-8704; and introduction of Pro residues in position A435 and S436 (Li et al., 1997, Prot. Eng. 10: 1199-1204.

Other glucoamylases include Athelia rolfsii (previously denoted Corticium rolfsii) glucoamylase (see U.S. Pat. No. 4,727,026 and (Nagasaka. et al., 1998, “Purification and properties of the raw-starch-degrading glucoamylases from Corticium rolfsii, Appl. Microbiol. Biotechnol. 50: 323-330), Talaromyces glucoamylases, in particular derived from Talaromyces emersonii (WO 99/28448), Talaromyces leycettanus (U.S. Pat. No. Re. 32,153), Talaromyces duponti, Talaromyces thermophilus (U.S. Pat. No. 4,587,215).

Bacterial glucoamylases contemplated include glucoamylases from the genus Clostridium, in particular C. thermoamylolyticum (EP 135,138), and C. thermohydrosulfuricum (WO 86/01831) and Trametes cingulata, Pachykytospora papyracea; and Leucopaxillus giganteus all disclosed in WO 2006/069289; or Peniphora rufomarginata disclosed in PCT/US2007/066618; or a mixture thereof. Also hybrid glucoamylase are contemplated. Examples the hybrid glucoamylases disclosed in WO 2005/045018. Specific examples include the hybrid glucoamylase disclosed in Table 1 and 4 of Example 1 (which hybrids are hereby incorporated by reference).

### Hydrolysis of Lignocellulosic Biomass

Conversion of lignocellulose-containing material into fermentation products, such as ethanol, has the advantages of the ready availability of large amounts of feedstock, including wood, agricultural residues, herbaceous crops, municipal solid wastes etc. Lignocellulose-containing materials primarily consist of cellulose, hemicellulose, and lignin and are often referred to as “biomass”.

The structure of lignocellulose is not directly accessible to enzymatic hydrolysis. Therefore, the lignocellulose-containing material is preferably pre-treated, e.g., by acid hydrolysis under adequate conditions of pressure and temperature, in order to break the lignin seal and disrupt the crystalline structure of cellulose, so as to cause solubilization of the hemicellulose and cellulose fractions. The cellulose and hemicelluloses can then be hydrolyzed enzymatically, e.g., by cellulolytic enzymes, to convert the carbohydrate polymers into fermentable sugars which may be fermented into desired fermentation products, such as ethanol. Optionally the fermentation product may be recovered, e.g., by distillation.

Thus, the process of producing a fermentation product from lignocellulose-containing material may comprise the steps of:

(a) pre-treating lignocellulose-containing material;

(b) hydrolyzing the material;

(c) fermenting with a fermenting organism.

Hydrolysis step (b) and fermentation step (c) may be carried out sequentially or simultaneously. In preferred embodiments the steps are carried out as SHF or HHF process steps which will be described further below.

### Pre-Treatment

The lignocellulose-containing material may be pre-treated before being hydrolyzed and/or fermented. In a preferred embodiment the pre-treated material is hydrolyzed, preferably enzymatically, before and/or during fermentation. The goal of pre-treatment is to separate and/or release cellulose, hemicellulose and/or lignin and this way improve the rate of enzymatic hydrolysis.

Pre-treatment step (a) may be a conventional pre-treatment step known in the art. Pre-treatment may take place in aqueous slurry. The lignocellulose-containing material may during pre-treatment be present in an amount between 10-80 wt. %, preferably between 20-50 wt. %.

### Hydrolysis and Fermentation

Hydrolysis and fermentation can be carried out as a simultaneous hydrolysis and fermentation step (SSF). In general this means that combined/simultaneous hydrolysis and fermentation are carried out at conditions (e.g., temperature and/or pH) suitable, preferably optimal, for the fermenting organism(s) in question.

Hydrolysis and fermentation can also be carried out as hybrid hydrolysis and fermentation (HHF). HHF typically begins with a separate partial hydrolysis step and ends with a simultaneous hydrolysis and fermentation step. The separate partial hydrolysis step is an enzymatic cellulose saccharification step typically carried out at conditions (e.g., at higher temperatures) suitable, preferably optimal, for the hydrolyzing enzyme(s) in question. The subsequent simultaneous hydrolysis and fermentation step is typically carried out at conditions suitable for the fermenting organism(s) (often at lower temperatures than the separate hydrolysis step).

Hydrolysis and fermentation can also be carried out as separate hydrolysis and fermentation, where the hydrolysis is taken to completion before initiation of fermentation. This is often referred to as “SHF”.

### Cellulase

When the plant material comprises lignocellulosic biomass, the enzyme may comprise cellulase and/or hemicellulase. The cellulase may comprise cellobiohydrolases (EC 3.2.1.91), e.g., cellobiohydrolase I and cellobiohydrolase II, as well as endo-glucanases (EC 3.2.1.4) and beta-glucosidases (EC 3.2.1.21). particularly endo-glucahase I and/or II (EG-I, EG-II), cellobiohydrolase I and/or II (CBH-I CBH-II) and/or beta-glucosidase.

For efficient digestion of cellulose and hemicelluloses, several types of enzymes acting cooperatively should be used, generally including at least three categories of enzymes in order to convert cellulose into fermentable sugars: endo-glucanases (EC 3.2.1.4) which cut the cellulose chains at random; cellobiohydrolases (EC 3.2.1.91) which cleave cellobiosyl units from the cellulose chain ends and beta-glucosidases (EC 3.2.1.21) which convert cellobiose and soluble cellodextrins into glucose. Among these three categories of enzymes involved in the biodegradation of cellulose, cellobiohydrolases are the key enzymes for the degradation of native crystalline cellulose. The term “cellobiohydrolase I” is a cellulose 1,4-beta-cellobiosidase (also referred to as Exo-glucanase, Exo-cellobiohydrolase or 1,4-beta-cellobiohydrolase) activity, as defined in the enzyme class EC 3.2.1.91, which catalyzes the hydrolysis of 1,4-beta-D-glucosidic linkages in cellulose and cellotetraose, by the release of cellobiose from the non-reducing ends of the chains. The definition of the term “cellobiohydrolase II activity” is identical, except that cellobiohydrolase II attacks from the reducing ends of the chains.

Endoglucanases (EC No. 3.2.1.4) catalyse endo hydrolysis of 1,4-beta-D-glycosidic linkages in cellulose, cellulose derivatives (such as carboxy methyl cellulose and hydroxy ethyl cellulose), lichenin, beta-1,4 bonds in mixed beta-1,3 glucans such as cereal beta-D-glucans or xyloglucans and other plant material containing cellulosic parts. The authorized name is endo-1,4-beta-D-glucan 4-glucano hydrolase, but the abbreviated term endoglucanase is used in the present specification.

The cellulase activity may, in a preferred embodiment, be derived from a fungal source, such as a strain of the genus Trichoderma, preferably a strain of Trichoderma reesei; a strain of the genus Humicola, such as a strain of Humicola insolens; or a strain of Chrysosporium, preferably a strain of Chrysosporium lucknowense.

In a preferred embodiment the cellulase preparation comprises a polypeptide having cellulolytic enhancing activity (GH61A), preferably the one disclosed in WO 2005/074656. The cellulase preparation may further comprise a beta-glucosidase, such as the fusion protein disclosed in U.S. 60/832,511. In an embodiment the cellulase preparation also comprises a CBH II, preferably Thielavia terrestris cellobiohydrolase II CEL6A. In an embodiment the cellulase preparation also comprises a cellulase enzymes derived from Trichoderma reesei. In a preferred embodiment the cellulase preparation is Cellulase preparation A used in Example 1 and disclosed in WO 2008/151079.

A cellulolytic enzyme may be added for hydrolyzing the pre-treated lignocellulose-containing material. The cellulolytic enzyme may be dosed in the range from 0.1-100 FPU per gram total solids (TS), preferably 0.5-50 FPU per gram TS, especially 1-20 FPU per gram TS. In another embodiment at least 0.1 mg cellulolytic enzyme per gram total solids (TS), preferably at least 3 mg cellulolytic enzyme per gram TS, such as between 5 and 10 mg cellulolytic enzyme(s) per gram TS is(are) used for hydrolysis. The FPU unit is defined in WO 2009/052500.

### Hemicellulase

Any hemicellulase suitable for use in hydrolyzing hemicellulose, preferably into xylose, may be used. Preferred hemicellulases include xylanases, arabinofuranosidases, acetyl xylan esterase, feruloyl esterase, glucuronidases, endo-galactanase, mannases, endo or exo arabinases, exo-galactanses, and mixtures of two or more thereof. Preferably, the hemicellulase for use in the present invention is an exo-acting hemicellulase, and more preferably, the hemicellulase is an exo-acting hemicellulase which has the ability to hydrolyze hemicellulose under acidic conditions of below pH 7, preferably pH 3-7. An example of hemicellulase suitable for use in the present invention includes VISCOZYME™ (available from Novozymes NS, Denmark).

In an embodiment the hemicellulase is a xylanase. In an embodiment the xylanase may preferably be of microbial origin, such as of fungal origin (e.g., Trichoderma, Meripilus, Humicola, Aspergillus, Fusarium) or from a bacterium (e.g., Bacillus). In a preferred embodiment the xylanase is derived from a filamentous fungus, preferably derived from a strain of Aspergillus, such as Aspergillus aculeatus; or a strain of Humicola, preferably Humicola lanuginosa. The xylanase may preferably be an endo-1,4-beta-xylanase, more preferably an endo-1,4-beta-xylanase of GH10 or GH11. Examples of commercial xylanases include SHEARZYME™ and BIOFEED WHEAT™ from Novozymes NS, Denmark.

The hemicellulase may be added in an amount effective to hydrolyze hemicellulose, such as, in amounts from about 0.001 to 0.5 wt. % of total solids (TS), more preferably from about 0.05 to 0.5 wt. % of TS.

Xylanases may be added in amounts of 0.001-1.0 g/kg DM (dry matter) substrate, preferably in the amounts of 0.005-0.5 g/kg DM substrate, and most preferably from 0.05-0.10 g/kg DM substrate.

### EXAMPLES

### Example 1

In this example, glucoamylase and alpha-amylase are dosed as described in WO 2008/141133, Example 1. 1.5 kg of a spray-dried enzyme composition comprising 1 AGU/g and 0.1625 FAU-F/g or comprising 30-50% enzyme protein is packed in a paper bag and sealed. The AGU and FAU-F assays are described in WO 2008/141133.

A slurry is formed by adding 10000 kg of ground yellow dent corn (with an average particle size around 0.5 mm) to to 15000 kg tap water in a 40 m³ fermenter vessel (equipped with stirrer blades). This mixture is supplemented with 75 L 1 g/L penicillin and 25 kg of urea. The pH of this slurry is adjusted to 4.5 with NaOH (initial pH before adjustment is about 3.8). The dry solids (DS) of the slurry is 35% wt. This slurry is dosed with the 1.5 kg spray-dried enzyme (equal to 0.4 AGU +0.065 FAU per g DS) in a sealed paper bag, by adding the bag directly into the slurry. The slurry is stirred for 120 minutes to allow for the disintegration of the paper bag and the enzyme to work on the substrates.

500 L yeast propagate is added to the slurry. Simultaneous saccharification and fermentation is performed at 32° C. for 70 hours followed by recovery of the ethanol using a suitable method known to the person skilled in the art.

### Example 2

In this example, a cellulase preparation is dosed as described in WO 2009/003167, Example 1. 1000 kg spray dried enzyme powder comprising cellulase preparation A described in WO 2009/003167 is packed in sealed cardboard boxes, each containing 25 kg enzyme powder. The spray-dried powder comprises approx. 50-80% w/w enzyme protein.

Corn stover is pretreated in a process according to NREL (TP-510-32438, June 2002). A 3000 tons slurry having 20% dry solids is formed from pretreated corn stover (PCS) and water. The slurry is heated to 50° C. and fed to a 3596 m³ saccharification vessel equipped with stirrers. 1000 kg of the spray dried cellulase powder and packed in sealed 25 kg cardboard boxes is added directly into the slurry. The enzyme dosage in relation to the substrate dry solids (DS) is in the range 1-20 FPU per gram DS or 1-2500 mg EP (enzyme protein)/kg DS. The FPU assay is described in WO 2009/003167.

The boxes disintegrate in the PCS. The enzymes are allowed to hydrolyze the PCS for 36 hours at 50° C.

The slurry is subsequently cooled and fed to the fermenter, yeast is added, and fermentation is performed at 32° C. for 72 hours. Ethanol is recovered using a suitable method known to the person skilled in the art.

1-15. (canceled)

16. A process for hydrolyzing a plant material, comprising:
(a) preparing an aqueous solution or suspension of the plant material,
(b) adding one or more container(s) which enclose(s) a multitude of particles comprising one or more enzyme(s) having hydrolytic activity towards the plant material so that the enzyme(s) is/are released to the solution or suspension, wherein the one or more containers filled with particles has a mass of at least 20 kg, and
(c) incubating the solution or suspension so as to hydrolyze the plant material.

17. The process of claim 16, wherein the one or more containers filled with particles have a mass of at least 25 kg.

18. The process of claim 16, wherein the one or more containers filled with particles have a mass of at least 100 kg.

19. The process of claim 16, wherein the one or more containers filled with particles have a mass of at least 500 kg.

20. The process of claim 16, wherein the material of the one or more containers comprises water soluble or dispersible packaging material.

21. The process of claim 16, wherein the material of the one or more containers comprises paper, cardboard, polyvinyl alcohol, water soluble cellulose derivatives, or water soluble starch derivatives.

22. The process of claim 16, wherein the material of the one or more containers comprises paper or cardboard.

23. The process of claim 16, wherein the one or more containers are paper bags in cardboard boxes.

24. The process of claim 16, wherein the particles are made by spray-drying and/or fluid bed drying.

25. The process of claim 16, wherein the particles have an average mass below 1 g.

26. The process of claim 16, wherein the container retains at least two kinds of particles comprising different enzymes.

27. The process of claim 16, wherein the incubation occurs in a tank equipped with agitation.

28. The process of claim 16, wherein the plant material comprises starch, and the one or more enzymes have alpha-amylase and/or glucoamylase activity.

29. The process of claim 28, wherein the particles comprise alpha-amylase at an activity of at least 0.02 Fungyl Amylase Units (FAU-F)/g and/or glucoamylase at an activity of at least 0.1 Amyloglucosidase Units (AGU)/g.

30. The process of claim 16, wherein the plant material comprises lignocellulosic biomass, and the one or more enzymes comprise cellulase and/or hemicellulase.

31. The process of claim 16, wherein the particles comprise at least 1% by weight of enzyme protein.

32. A process for producing a fermentation product, which comprises a process for hydrolyzing a plant material of claim 16 and adding a fermenting organism and incubating so as to form a fermentation product during or after step c).

33. The process of claim 32, wherein the fermentation product is ethanol.
